(19) United States
(12) Patent Application Publication
(10) Pub. No.: US 2006/0128696 A1
(43) Pub. Date: Jun. 15, 2006

(54) Treating seizures using ice inhibitors

(76) Inventors: Annamaria Vezzani, Milano (IT); John Randle, Brookline, MA (US)

Correspondence Address: VERTEX PHARMACEUTICALS INC., 130 WAVERLY STREET, CAMBRIDGE, MA, 02139-4242, US

(21) Appl. No.: 11/130,659

(22) Filed: May 16, 2005

Related U.S. Application Data
(60) Provisional application No. 60/571,314, filed on May 15, 2004.

(51) Int. Cl. A61K 31/5513

(52) U.S. Cl. 514/221

(57) ABSTRACT

This invention relates to methods and compositions for treating or preventing seizures.

### CROSS-REFERENCE TO RELATED APPLICATIONS

The present application claims the benefit under 35 U.S.C. § 119 of U.S. Provisional patent application No. 60/571,314, filed May 15, 2004, the entire contents of the application being incorporated herein by reference.

### FIELD OF THE INVENTION

This invention relates to methods and compositions for treating or preventing seizures with an ICE inhibitor.

### BACKGROUND OF THE INVENTION

Cytokines (especially IL-1β and TNF-α) are optimum therapeutic targets as they can initiate and sustain many diseases. Various strategies such as soluble receptors, antibodies, receptor antagonists or inhibitors are used to block cytokines. These specific anti-cytokine-based therapies have been shown to reduce inflammation in many chronic inflammatory or autoimmune diseases and are approved by FDA for human use (Bresnihan et al., 1998; Mohler et al., 1993; Nuki et al., 2002; van Deventer, 1999).

Interleukin-1β converting enzyme (ICE, also known as caspase-1), is an intracellular protease that cleaves the precursors of IL-1β and IL-18 into active cytokines (Akita et al., 1997; Kuida et al., 1995). Although other proteases (including bacterial and host proteases) can process pro-IL-1β, ICE-deficient (ICE^{-/-}) mice have been shown incapable of releasing mature IL-1β in response to endotoxin [Fantuzzi et al., 1997; Li et al., 1995].

Expression of proinflammatory and Anti-inflammatory Cytokines in the Brain have been Linked to Seizures. [A. Vezzani et al., “Interleukin-1β Immunoreactivity and Microglia Are Enhanced in the Rat Hippocampus by Focal Kainate Application: Functional Evidence for Enhancement of Electrographic Seizures” J. Neurosci., 19, pp. 5054-5065 (1999); DeSimoni et al., “Inflammatory cytokines and related genes and are induced in the rat hippocampus by limbic status epilepticus” Eur. J. Neurosci., 12, pp. 2623-2633 (2000); A. Vezzani et al., “Powerful Anticonvulsant Action of IL-Receptor Antagonist on Intracerebral Injection and Astrocytic Overexpression in Mice” PNAS, 97, pp. 11534-11539 (2000)]. However, there are currently no acceptable anti-cytokine or anti-inflammatory drugs for use as anti-convulsant or anti-epilepsy therapies.

### SUMMARY OF THE INVENTION

The present invention relates to methods for treating or preventing seizures, convulsions, epilepsy, and related conditions by administering an ICE inhibitor.

The present invention also relates to compounds and compositions for treating or preventing seizures, convulsions, epilepsy or related conditions.

The present invention also relates to methods for identifying agents useful for treating or preventing such conditions.

The invention also relates to processes for preparing compositions and kits for practicing a method of this invention.

### BRIEF DESCRIPTION OF THE FIGURES

FIG. 1 depicts the effect of compound 1 (25 μg in 4 μl icv) on caspase-1 levels (assessed by western blotting) in the hippocampus of kainic acid-treated rats. Rats were killed 90 min after the beginning of EEG seizures induced by intrahippocampal microinjection of 40 ng kainic acid (see also, FIG. 2A and FIG. 2B).

FIG. 2A and FIG. 2B represent the results of a Western blot analysis of ICE/caspase-1 and IL-1β levels in sham hippocampi and 90 minutes after kainic acid-induced seizures, with or without compound 1 treatment. FIG. 2A and FIG. 2B are histogram representations of the Western blot data, illustrated as the mean±SEM from 4 rats. Compound 1 (25 μg/4 μL) or vehicle were injected intracerebroventricularly 45 and 10 min before intrahipocampal injection of kainic acid (40 ng). Compound 1 blocked the seizure-induced production of the mature form of caspase-1 (see also FIG. 1) and of the mature form of IL-1β. *p<0.05; **p<0.001 by Tukey's test. See Example 1 and Example 6.

### DETAILED DESCRIPTION OF THE INVENTION

This invention provides methods for treating or preventing seizures by administering an ICE inhibitor in an amount effective for treating or preventing seizures.

Applicants have demonstrated that the use of an ICE inhibitor is effective at treating seizures in rodents. Specifically, applicants have demonstrated that treatment with an ICE inhibitor increases the time to onset of seizures and decreases the time spent in seizures. The ICE inhibitor compound 1 was as effective as high doses of either phenyloin or carbamazepine, which are known anticonvulsant compounds.

Accordingly, one embodiment of this invention provides therapeutic strategies for inhibiting seizures. These methods may be used to regulate, ameliorate, treat, or prevent seizures. The methods could also be used to ameliorate, treat, or prevent the progession and worsening of a seizure disorder. Such methods would involve, for example, administering an ICE inhibitor following traumatic brain injury, infection, or febrile seizure event to prevent or lessen the severity of a permanent seizure disorder.

Other embodiments of this invention provide therapeutic strategies for regulation, ameliorating, treating, or preventing epilepsy, convulsions, and related disorders.

Applicants have also shown that compound 1 and compound 2 inhibit seizures when administered by the intraperitoneal route (Table 3).

The ICE inhibitor compounds are known for their anti-inflammatory activity in animal models of rheumatoid arthritis, dermatological inflammatory disease and inflammatory bowel disease, among others [G. Ku et al., “Selective Interleukin-1 Converting Enzyme (ICE/Caspase-1) Inhibition With Pralnacasan (HMR 3480/VX-740) Reduces Inflammation and Joint Destruction in Murine Type-II Collagen-induced Arthritis (CIA)” American College of Rheumatology, San Francisco, Nov. 12-15, 2001; G. Ku et al. “Interleukin-1β Converting Enzyme (ICE, Caspase-1) Inhibition with VX-765 Reduces Inflammation and Cytokine Levels in Murine Dermatitis and Arthritis Models” International Congress of Immunology, Stockholm, Sweden, Jul. 22-27, 2001; G. Ku et al. “Interleukin-1β Converting Enzyme (ICE, Caspase-1) Inhibition with VX-765 Reduces Inflammation and Cytokine Levels in Murine Oxazolone-induced Dermatitis” The Society for Investigative Dermatology, May 9-12, 2001 Abstract # 856; see also ICE inhibitor documents cited herein]. Compound 1 has also been demonstrated to have anti-inflammatory activity in rheumatoid arthritis patients [K. Pavelka et al., “Clinical Effects of Pralnacasan (PRAL), an Orally-active Interleukin-1β Converting Enzyme (ICE) Inhibitor, in a 285 Patient PHII Trial in Rheumatoid Arthritis (RA)” American College of Rheumatology 2002 Conference Late-Breaking Abstract, New Orleans, Oct. 25-29, 2002]. ICE inhibitors have not been used to treat seizures ro seizure disorders.

The pharmacokinetics of these compounds underlying their anti-inflammatory activity in animals and humans is well-understood. Furthermore, applicants have observed that these compounds penetrate into the brain, albeit at considerably lower concentrations than in the blood and certain peripheral tissues. This latter characteristic is presumed to be essential to the activity of any anti-convulsant or anti-epileptic agent and it is unclear whether the brain concentrations attained by the compounds are sufficient to inhibit ICE/caspase-1 in the brain and inhibit IL-1β production and its contribution to seizure development. Applicants have demonstrated nevertheless that compound 1 and compound 2 have anti-convulsant activity when administered peripherally.

The advantageous effects of ICE inhibitors on seizures is not directly related to the antinflammatory activity of the ICE inhibitors. Ibuprofen, a known anti-inflammatory agent, was tested in applicants' seizure model, administered by the intraperitoneal route. Ibuprofen increased the seizure activity compared to vehicle (see Table 4). Relative to vehicle, ibuprofen increased the time in status epilepticus, thus indicating that ibuprofen increases or induces seizure activity.

The examples provided herein involve an rodent seizure model that is recognized as a good model of human epilepsy and convulsions disorders. For example, known anti-epileptic drugs such as carbamazepine and phenyloin exhibit anti-convulsant activity in this model, as do the ICE inhibitors.

Although the applicants have studied the anti-convulsant activity of the compounds following their intracerebroventricular and intraperitoneal administration, prior experience with compound 1 and compound 2 administered by a variety of peripheral routes, including intraperitoneal, oral and intravenous, indicates that the compounds would also have anticonvulsant activity when administered by these alternate routes. In a preferred embodiment, the ICE inhibitor is administered peripherally (i.e., orally or parenterally, not intracranially).

The present invention involves the use of compounds that are inhibitors of ICE. Such compounds may be selective for ICE. Or such compounds may be active against ICE and active against another caspase or against a range of other caspases (e.g., 2-14). As demonstrated herein, inhibiting ICE and inhibiting IL-1β production will delay the time to onset of seizures, decrease the amount of time spent in seizures, or decrease the frequency of seizures, including any one or more or all of the above. The data generated in Example 1 and Example 6 demonstrate that anticonvulsant doses of compound 1 have the expected mechanism-related effects on ICE/caspase-1 activation and IL-1β production.

In the methods of this invention, a compound would be administered in an amount effective to inhibit ICE and to therefore treat seizures (or other related disorders). Treating seizures (or other related disorders) includes reducing the duration of a seizure, reducing the severity of a seizure, reducing susceptibility of seizure onset, delaying seizure onset, eliminating the occurrence of a seizure. Therefore, also provided by this invention are methods for preventing seizures (or other related disorders) by administering and ICE inhibitor in an amount effective for preventing seizures.

The methods of this invention may be used to treat animals, preferably mammals, including human and non-human mammals. Any compound that inhibits ICE may be used in the methods and compositions of this invention. Such compounds include those compounds that inhibit ICE selectively and those that inhibit one or more enzyme in the caspase or ICE/CED-3 family. Compounds for use in connection with this invention inhibit the catalytic activity of ICE in either a reversible or irreversible manner.

The compounds of this invention inhibit ICE and/or decrease IL-1, particularly IL-1β and IL-18 levels. These compounds can be assayed, for example, for their ability to inhibit ICE, the production of IL-1β and/or IL-18, the regulation of IL-1 and/or IL-18 levels, and/or affect IL-1β and/or IL-18 activity. Assays for testing each of these activities are known in the art (see Examples herein, WO 95/35308, WO 97/22619, WO 99/47545, or WO 01/90063). Accordingly, these compounds are capable of targeting and inhibiting events in the ICE and/or IL-1 mediated diseases set forth herein.

Compounds that may be used in connection with this invention include, but are not limited to, the compounds of the following documents: WO 04/058718, WO 04/002961, WO 03/088917, WO 03/068242, WO 03/042169, WO 98/16505, WO 93/09135, WO 03/106460, WO 03/103677, WO 03/104231, WO 02/085899, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA, WO 02/22611, US 2002/0058630, WO 02/12638, WO 95/35308, U.S. Pat. No. 5,716,929, WO 97/22619, U.S. Pat. No. 6,204,261, WO 99/47545, WO 01/90063, US Patent Publication 2004/0014753, US Patent Publication 2004/0009966, US Patent Publication 2003/0236296, U.S. Pat. No. 6,693,096, U.S. Pat. No. 6,610,683, U.S. Pat. No. 6,531,467, U.S. Pat. No. 6,528,506, U.S. Pat. No. 6,200,969, WO 2003/072528, WO 2003/032918, WO 01/00658, WO 98/10778, U.S. Pat. No. 6,716,818, U.S. Pat. No. 6,620,782, U.S. Pat. No. 6,566,338, U.S. Pat. No. 6,495,522, U.S. Pat. Nos. 6,355,618, 6,153,591, WO 2005/003100, WO 2004/002401, WO 00/61542, WO 00/55114, WO 99/47154, U.S. Pat. No. 6,083,981, U.S. Pat. No. 5,932,549, U.S. Pat. No. 5,919,790, U.S. Pat. No. 5,744,451, WO 2002/089749, WO 99/36426, WO 98/16505, WO 98/16504, WO 98/16502, U.S. Pat. No. 6,316,415, U.S. Pat. No. 5,932,549, U.S. Pat. No. 5,919,790, U.S. Pat. No. 5,744,451, EP 1082127, EP 1049703, EP 0932600, EP 0932598, WO 99/56765, WO 93/05071, EP 0600880, and EP 1378573 (which, as set forth herein, are all incorporated by reference herein). Preferred compounds for use in this invention include those of WO 04/058718, WO 04/002961, WO 95/35308, WO 97/22619, WO 99/47545, and WO 01/90063. Other preferred compounds for use in this invention include those of WO 95/35308, WO 97/22619, WO 99/47545, and WO 01/90063. More preferred compounds are those recited in the claims herein. These compounds may be obtained by methods known to skilled practitioners and the methods disclosed in documents cited herein.

This invention also provides assays for testing compounds for anti-seizure, anti-epileptic, or anti-convulsant activity according to the methods herein. Such methods involve, for example, identifying a compound useful in the treatment of seizures, convulsions, epilepsy, or related disorders comprising determining the ability of the compound to inhibit ICE and/or to inhibit seizures, convulsions, epilepsy, or related disorders. Other methods of this invention involve assaying ICE inhibitors for anticonvulsant activity. Such methods and assays are useful for identifying a compound for use in the treatment of seizures, convulsions, epilepsy, or related disorders. In preferred embodiments, the assays may be done by methods substantially as described herein (see, e.g., Examples 1, 2, or 3).

The pharmaceutical compositions and methods of this invention, therefore, will be useful for controlling IL-1 levels and/or activity in vitro or in vivo. The compositions and methods of this invention will thus be useful for controlling IL-1 levels in vivo and for treating or reducing the advancement, severity or effects of certain conditions, including diseases, disorders, or effects as set forth herein.

According to another embodiment, the invention provides a composition comprising a compound of this invention or a pharmaceutically acceptable derivative (e.g., salt) thereof, as described above, and a pharmaceutically acceptable carrier.

According to another embodiment, the compositions and methods of this invention may further comprise another therapeutic agent. Such agents include, but are not limited to, a compound for treating or inhibiting seizures, convulsions, or epilepsy, such as a barbiturate (e.g., mephobarbital, pentobarbital), a benzodiazepine (e.g., lorazepam clonazepam, clorazepate, diazepam), a GABA analogue (e.g., tiagabin, gabapentin, pregabalin, vigabatrin), a hydantoins (e.g., phenyloin, fosphenyloin) a phenyltriazine (e.g., lamotrigine), a succinimide (e.g, methsuximide, ethosuximide) or other, miscellaneous compounds (e.g., carbamazepine, riluzole, valproate, divalproex, felbamate, primidone, or topiramate), an anti-inflammatory agent, a matrix metalloprotease inhibitor, a lipoxygenase inhibitor, a cytokine antagonist, an immunosuppressant, an anti-cancer agent, an anti-viral agent, a cytokine, a growth factor, an immunomodulator (e.g., bropirimine, anti-human alpha interferon antibody, IL-2, GM-CSF, methionine enkephalin, interferon alpha, diethyldithiocarbamate, tumor necrosis factor, naltrexone and rEPO), a prostaglandin, or an anti-vascular hyperproliferation compound.

The term “pharmaceutically acceptable carrier” refers to a non-toxic carrier that may be administered to a patient, together with a compound of this invention, and which does not destroy the pharmacological activity thereof.

Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In pharmaceutical compositions comprising only a compound of this invention as the active component, methods for administering these compositions may additionally comprise the step of administering to the subject an additional agent. Such agents include, but are not limited to, a compound for treating or inhibiting seizures, convulsions, or epilepsy, such as barbiturate (e.g., mephobarbital, pentobarbital), a benzodiazepines (e.g., lorazepam clonazepam, clorazepate, diazepam), a GABA analogue (e.g., tiagabin, gabapentin, pregabalin, vigabatrin), a hydantoins (e.g, phenyloin, fosphenyloin) a phenyltriazine (e.g., lamotrigine), a succinimide (e.g, methsuximide, ethosuximide) or other, miscellaneous compounds (e.g., carbamazepine, riluzole, valproate, divalproex, felbamate, primidone, or topiramate), an anti-inflammatory agent, a matrix metalloprotease inhibitor, a lipoxygenase inhibitor, a cytokine antagonist, an immunosuppressant, an anti-cancer agent, an anti-viral agent, a cytokine, a growth factor, an immunomodulator (e.g., bropirimine, anti-human alpha interferon antibody, IL-2, GM-CSF, methionine enkephalin, interferon alpha, diethyldithiocarbamate, tumor necrosis factor, naltrexone and rEPO), a prostaglandin, or an anti-vascular hyperproliferation compound. When a second agent is used, the second agent may be administered either as a separate dosage form or as part of a single dosage form with the compounds or compositions of this invention.

The amount of compound present in the above-described compositions should be sufficient to cause a detectable decrease in the severity of the disease, or in ICE inhibition, IL-1 levels, or IL-1 activity.

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this invention are formulated for pharmaceutical administration to a subject, e.g., a mammal, preferably a human being.

Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection and infusion techniques. Preferably, the compositions are administered orally.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil and castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

If a solid carrier is used, the preparation can be tableted, placed in a hard gelatin capsule in powder or pellet form, or in the form of a troche or lozenge. The amount of solid carrier will vary, e.g., from about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

A syrup formulation can consist of a suspension or solution of the compound in a liquid carrier for example, ethanol, glycerin, or water with a flavoring or coloring agent. An aerosol preparation can consist of a solution or suspension of the compound in a liquid carrier such as water, ethanol or glycerin; whereas in a powder dry aerosol, the preparation can include e.g., a wetting agent.

Formulations of the present invention comprise an active ingredient together with one or more acceptable carrier(s) thereof and optionally any other therapeutic ingredient(s). The carrier(s) should be “acceptable” in the sense of being compatible with the other ingredients of the formulation and not deleterious to the recipient thereof.

The pharmaceutical compositions of this invention may be orally administered in any orally acceptable dosage form including, but not limited to, capsules, tablets, and aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, (including e.g., during intracranial surgery). Suitable topical formulations are readily prepared for each of these applications.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. In one embodiment, the compositions are as formulated herein. Other ophthalmic preparations may be found in, e.g., U.S. Pat. No. 6,645,994 and/or U.S. Pat. No. 6,630,473.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents known in the art.

It will be recognized by one of skill in the art that the form and character of the pharmaceutically acceptable carrier or diluent is dictated by the amount of active ingredient with which it is to be combined, the route of administration, and other well-known variables.

The above-described compounds and compositions are also useful in therapeutic applications relating to certain diseases associated with seizures or convulsions.

The compounds of this invention can inhibit the release of IL-1β and/or IL-18 and thus can be useful for inhibiting or blocking several pathophysiological effects of certain diseases as set forth herein.

This invention also relates to a therapeutic method for treating certain diseases by (1) inhibiting IL-1 release from cells and/or (2) preventing the untoward, toxic or lethal effects of excessively high tissue levels of IL-1 in a mammal, including a human. This method comprises administering to a mammal an effective ICE inhibiting quantity of one or more ICE/CED-3 inhibitors. This method also can be used for the prophylactic treatment or prevention of certain diseases amenable thereto, including seizures, convulsions, epilepsy, or related disorders. The invention provides a method for the treating these disorders by administering to a mammal, including a human, in need thereof an effective amount of such compounds.

The compounds, by inhibiting ICE and blocking the release of IL-1 or decreasing IL-1 levels and activity, as well as the pathophysiologic actions of excessive levels of IL-1 in each of these circumstances, directly facilitate the arrest or resolution of certain diseases, and facilitates the restoration of normal function. Together, these actions relate their novel use in treating seizures and related disorders.

ICE inhibition may be measured by methods known in the art and as described more fully herein.

The compounds may be useful in inhibiting the release of IL-1 release by monocytes, macrophages, neuronal cells, endothelial cells, epidermal cells, mesenchymal cells (for example: fibroblasts, skeletal myocytes, smooth muscle myocytes, cardiac myocytes) and many other types of cells.

The term “condition” or “state” refers to any disease, disorder, or effect that produces deleterious biological consequences in a subject.

The term “seizure” as used herein refers generically to sudden and involuntary contractions of muscles over the whole or part of the body, which contractions are caused by an abnormal excitation of subsets of neurons in the central nervous system. Seizures are the symptoms of epilepsy. The motor manifestation of seizures are accompanied by alterations of the electroencephalogram (EEG). These alterations may occur also in the absence of obvious motor manifestations.

The level of IL-1 protein in the blood or cell of a patient or a cell culture (i.e., within the cell or the cell culture media) can be determined by for example, assaying for immunospecific binding to IL 1 or to other proteins known to be produced as a result of the presence of active IL-1. Such methods are known in the art. For example, immunoassays which can be used include, but are not limited to competitive and non-competitive assay systems, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis with labeled antibodies. Such assays well known in the art (see, e.g., Ausubel et al, eds., 1994, Current Protocols in Molecular Biology, Vol. 1, John Wiley & Sons, Inc., New York, which is incorporated by reference herein in its entirety).

Competitive binding assays can also be used to determine the level of IL-1. One example of a competitive binding assay is a radioimmunoassay comprising the incubation of labeled proteins from cells expressing IL-1 (e.g., ³H or ¹²⁵I) with an IL-1 antibody in the presence of increasing amounts of unlabeled IL-1, and the detection of the IL-1 antibody bound to the labeled IL-1. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

IL-1 levels can also be assayed by activity, for example, IL-1 levels can be assayed by a cell line that is capable of detecting bioactive levels of cytokines like IL-1 or a growth factor. According to one embodiment, the levels of bioactive IL-1 in a biological sample is detected by incubating a cell line genetically engineered with isopropyl-b-D-thiogalactopyranoside. The cell line is incubated with the sample to be tested and cell death in the cell line is monitored by determining the intensity of blue color, which is indicative of a bioactive cytokine or growth factor in the sample tested. See also, e.g., Burns (1994) 20(1):40-44 for IL-1 activity assay of serum of patients.

Dosage levels of between about 0.01 and about 100 mg/kg body weight per day, preferably between about 0.5 and about 75 mg/kg body weight per day and most preferably between about 1 and about 50 mg/kg body weight per day of the active ingredient compound are useful in a monotherapy. Dosages of about 50 mg/kg to about 200 mg/kg have been tested and found to be effective (see Examples herein). For intracranial administration, dosage levels of between 1 ng and 1 g and preferably between 100 ng and 100 mg of the active ingredient compound are useful.

Typically, the pharmaceutical compositions of this invention will be administered from about 1 to 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

When the compositions of this invention comprise a combination of a compound of this invention and one or more additional therapeutic agents, both the compound and the additional agent should be present at dosage levels of between about 10% to about 80% of the dosage normally administered in a monotherapy regime.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. When the symptoms have been alleviated to the desired level, it may be possible to cease treatment. Patients may, however, require intermittent treatment on a long-term basis upon any recurrence or disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular compound and other therapeutic agent, if present, in the composition.

Accordingly, a method for treating or preventing a disease of this invention in a subject comprises the step of administering to the subject any compound, pharmaceutical composition, or combination described herein.

In a preferred embodiment, the invention provides a method of treating a mammal, having one of the aforementioned diseases, comprising the step of administering to said mammal a pharmaceutically acceptable composition described above. In this embodiment, if the patient is also administered another therapeutic agent, it may be delivered together with the compound of this invention in a single dosage form, or, as a separate dosage form. When administered as a separate dosage form, the other therapeutic agent may be administered prior to, at the same time as, or following administration of a pharmaceutically acceptable composition comprising a compound of this invention.

The methods for identifying a compound or composition for treating a disease according to this invention include methods for screening of a plurality of compounds or compositions for their ability to ameliorate the effects of certain disease(s) and/or improve the condition of a patient having certain disease(s) of this invention. According to one embodiment of this invention, high throughput screening can be achieved by having cells in culture in a plurality of wells in a microtiter plate, adding a different compound or composition to each well and comparing the ICE inhibition and/or IL-1 levels and/or activity in each cell culture to the levels or activity present in a cell culture in a control well. Controls that are useful for the comparison step according to this invention include cells or subjects that have not been treated with a compound or composition and cells or subjects have been treated with a compound or composition that is known to have no effect on ICE inhibition or activity. According to one embodiment of this invention, the high throughput screening is automated so that the steps including the addition of the cells to the plate up to the data collection and analysis after addition of the compound or composition are done by machine. Instruments that are useful in the comparison step of this invention, e.g., instruments that can detect labeled objects (e.g., radiolabelled, fluorescent or colored objects) or objects that are themselves detectable, are commercially available and/or known in the art. Accordingly, compounds and compositions according to this invention that are useful for treating the certain disease disclosed herein can be quickly and efficiently screened.

All applications, patents and references disclosed herein are incorporated by reference. In order that this invention be more fully understood, the following preparative and testing examples are set forth. These examples are for the purpose of illustration only and are not to be construed as limiting the scope of the invention in any way.

### EXAMPLE 1

An experimental model of seizures in male adult Sprague-Dawley rats was induced by unilateral microinjection of kainic acid (40 ng in 0.5 μl) in the dorsal hippocampus of freely-moving rats using chronically-implanted cannulae and electrodes. Briefly, animals were deeply anesthetized using Equithesin (1% phenobarbital and 4% chloral hydrate; 3 ml/kg, i.p.). Bipolar nichrome wire insulated electrodes (60 μm) were implanted bilaterally into the dentate gyrus of the dorsal hippocampus (septal pole), and a guide cannula (22 gauge) was unilaterally positioned on top of the dura and glued to one of the depth electrodes for the intrahippocampal injection of kainic acid. The coordinates from bregma for implantation of the hippocampal electrodes were (in mm: nose bar −2.5, AP −3.5, L ±2.4 and 3 below dura mater).

An additional guide cannula was unilaterally positoned on top of the dura mater for intracerebroventricular injection of compounds (in mm, nose bar −2.5; AP −1; L+1.5). A ground lead was positioned over the nasal sinus and two screw electrodes were placed bilaterally over the parietal cortex. The electrodes were connected to a multipin socket (March Electronics, NY) and, together with the injection cannula, were secured to the skull by acrylic dental cement.

Compound 1 (25 μg/4 μl) or equal volume of vehicle was administered by intracerebroventricular injection. Seizures were recorded and quantified by EEG analysis based on the following parameters: 1) the time to onset of the first ictal episode, 2) the number of ictal episodes during the 3 hours of recording, and 3) the time spent in ictal activity reckoned by adding together the duration of each ictal event. Compound 1 treatment significantly increased the latency to onset of convulsions and reduced the number of ictal episodes and the total time spent in ictal activity (Table 1).

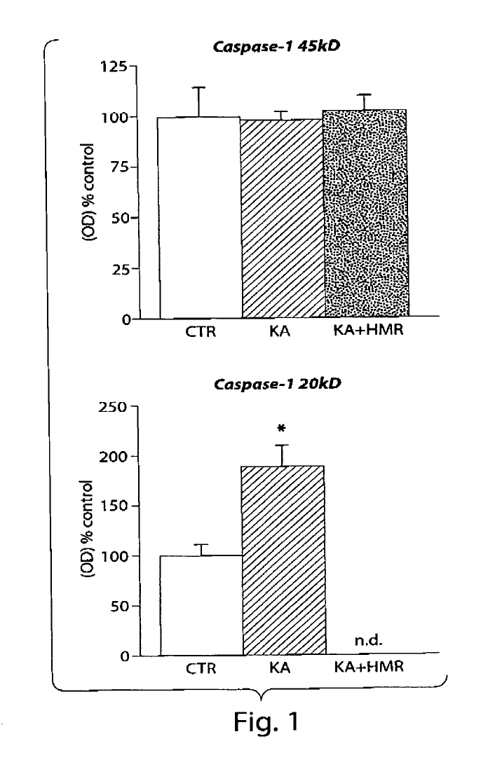

The effects of compound 1 on activation of ICE/caspase-1 was evaluated based on the amount of active 20 kD subunit detected by Western blot of samples from these rats. FIG. 1 shows that compound 1 treatment not only abolished the increase in the caspase-120 kD subunit induced by kainate seizures, but reduced this subunit to very low levels. The levels of the inactive 45 kD subunit of pro-caspase-1 were not changed by either kainate or compound 1.

TABLE 1
Rats received compound 1 (25 μg/4 μl) icv, 45 and 10 min before the injection of 40 ng in 0.5
μl kainic acid in the left hippocampus. Controls (vehicle) received 20% Cremophor in saline.

| Vehicle | | | | Compound 1 | | | |
|---|---|---|---|---|---|---|---|
| | | | Time in | | | | Time in |
| | | No. of | ictal | | | No. of | ictal |
| | ONSET | ictal | activity | | | ictal | activity |
| No RAT | (min) | episodes | (min) | N RAT | ONSET | episodes | (min) |
| 12 | 11.0 | 36 | 50.0 | 11 | 15.0 | 28 | 36.0 |
| 13 | 7.0 | 40 | 76.0 | 14 | 20.0 | 29 | 30.0 |
| 16 | 6.5 | 39 | 74.5 | 15 | 14.0 | 30 | 42.0 |
| 18 | 8.0 | 39 | 64.0 | 17 | 18.0 | 30 | 34.0 |
| 19 | 5.16 | 44 | 55.6 | 20 | 13.6 | 37 | 36.0 |
| 24 | 5.0 | 36 | 60.0 | 23 | 9.0 | 31 | 42.0 |
| 25 | 7.0 | 38 | 62.3 | 26 | 12.0 | 30 | 41.0 |
| 21 | 8.25 | 42 | Status | 22 | 14.0 | 33 | SE |
| Mean ± SE | 7.2 ± 0.7 | 39.2 ± 1 | 63.2 ± 3.6 | Mean ± SE | 14.4 ± 1.2** | 31.0 ± 1.0** | 37.3 ± 1.7** |

**p < 0.01 vs. vehicle by Student's t-test.

### EXAMPLE 2

An experimental model of seizures in rats was induced by unilateral microinjection of kainic acid (40 ng in 0,5 μL) in the dorsal hippocampus of freely-moving rats using chronically-implanted cannulae. Compound 1 (30 mg/kg) or vehicle was administered by intraperitoneal injection 45 and 10 min before kainic acid. EEG seizures were recorded using chronically-implanted hippocampal electrodes. Ictal and interictal epileptic activity was quantified by EEG analysis based on the following parameters: 1) the time to onset of the first ictal episode, 2) the number of ictal episodes during the 3 hours of recording, and 3) the time spent in ictal activity reckoned by adding together the duration of each ictal event. Compound 1 treatment significantly increased the latency to onset of convulsions and reduced the total time spent in ictal activity by ˜30% although this difference did not reach statistical significance (Table 2). These data suggest that a higher dose would be effective in producing a greater and statistically significant effect. See, Example 4, where a higher dose of compound 2 produced statistically significant effects.

TABLE 2
Rats received compound 1 (30 mg/kg) intraperitoneally, 45 and 10 min
before application of 40 ng in 0.5 μl kainic acid in the left hippocampus.
Control animals (vehicle) received 20% Cremophor in saline.

| Vehicle | | | | Compound 1 | | | |
|---|---|---|---|---|---|---|---|
| | | | Time in | | | | Time in |
| | | No. of | ictal | | | No. of | ictal |
| | ONSET | ictal | activity | | ONSET | ictal | activity |
| No RAT | (min) | episodes | (min) | No RAT | (min) | episodes | (min) |
| 1 | 6 | 17 | 16 | 2 | 10.5 | 8 | 9 |
| 3 | 3 | 18 | 47 | 4 | 12 | 23 | 32 |
| 5 | 5 | 35 | 26 | 6 | 14 | 29 | 27 |
| 7 | 7 | SE | SE | 8 | 9 | 17 | 20 |
| 9 | 9.1 | 15 | 72 | 10 | 9 | 23 | 34 |
| 11 | 10.5 | 21 | 35 | 12 | 23 | 25 | 25 |
| 13 | 11 | 20 | 32 | 14 | 13 | 16 | 30 |
| 15 | 9 | 20 | 28 | 16 | 11 | 25 | 28 |
| Mean ± SE | 7.5 ± 1.0 | 20.8 ± 2.5 | 36.6 ± 6.9 | Mean ± SE | 10.8 ± 0.7* | 20.5 ± 2.3 | 25.6 ± 2.8 |

*p < 0.01 vs. vehicle by Student's t-test.

### EXAMPLE 3

ICE Inhibition

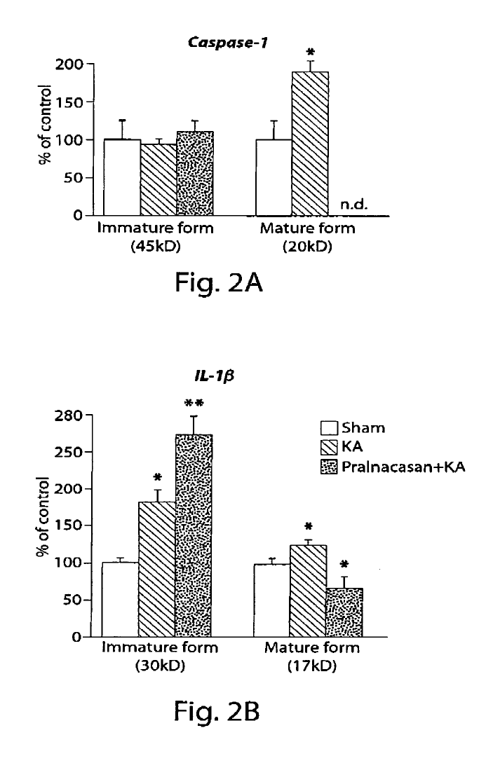

Compounds may be tested for their ability to inhibit ICE by methods known in the art (see, e.g., the documents cited in FIGS. 2-4).

### EXAMPLE 4

EEG seizures were induced in adult male Sprague-Dawley rats by intrahippocampal injection of 40 ng kainic acid (KA) using a chronically-implanted cannula. EEG seizures were recorded using chronically-implanted hippocampal electrodes. Ictal and interictal epileptic activity was quantified quantified by EEG analysis based on the following parameters: 1) the time to onset of the first ictal episode, 2) the number of ictal episodes during the 3 hours of recording, and 3) the time spent in ictal activity reckoned by adding together the duration of each ictal event. Compound 2 or its vehicle were injected intraperitoneally for 3 consecutive days (50-200 mg/kg). The 4^{th} day, rats received compound 2, 45 and 10 min before the intrahippocampal injection of 40 ng in 0.5 μl kainic acid.

TABLE 3
Effect of compound 2 on Kainate-induced Seizures in Rats

| | | | Number of | Time in |
|---|---|---|---|---|
| | Dose | Onset | ictal | ictal |
| Treatment | (mg/kg) | (min.) | episodes | activity |
| Vehicle | — | 8.5 ± 0.8 | 26.2 ± 1.5 | 25.5 ± 1.6 |
| Comp. 2 | 50 | 11.9 ± 0.7** | 15.6 ± 1.2** | 12.3 ± 3.3** |
| | 200 | 12.7 ± 0.8** | 19.7 ± 2.0** | 12.8 ± 1.3** |

Data are the mean ± SE (N = 7-15 rats).
**p < 0.01 vs. vehicle by one-way ANOVA followed by Dunnett's test.

### EXAMPLE 5

The effect of ibuprofen on seizures was also examined using the methods described in Example 4. Rats received ibuprofen (50 mg/kg, i.p.) 60 min. before unilateral intrahippocampal injection of 40 μg in 0.5 μl kainic acid. Controls (vehicle) received saline *p<0.05 vs. vehicle by Student's t-test. Seizures were analyzed and quantified by EEG. Status epilepticus represents continuous seizure activity lasting more than 30 min. consecutively.

TABLE 4
Vehicle

| | | | Time in | |
|---|---|---|---|---|
| | Onset | No. of | Seizures | Status |
| Rat | (min.) | Seizures | (min.) | Epilepticus |
| 1 | 11.6 | 13.0 | 16.0 | — |
| 2 | 7.5 | 16.0 | 18.5 | — |
| 3 | 21.0 | 20.0 | 21.0 | — |
| 4 | 10.0 | 15.0 | 23.0 | — |
| 5 | 21.0 | 20.0 | 21.0 | — |
| 6 | 10.0 | 15.0 | 23.0 | — |
| 7 | 11.6 | 17.0 | 25.0 | — |
| Mean ± SE | 13.2 ± 2.1 | 16.6 ± 1.0 | 21.1 ± 1.1 | — |

Ibuprofen

| | | | Time in | |
|---|---|---|---|---|
| | Onset | No. of | Seizures | Status |
| Rat | (min.) | Seizures | (min.) | Epilepticus |
| 1 | 14.4 | 13 | 13.0 | 75 |
| 2 | 7.9 | 10 | 8.4 | — |
| 3 | 11.0 | 13 | 11.0 | 66.6 |
| 4 | 12.3 | 12 | 12.5 | 80 |
| 5 | 13.3 | 16 | 11.2 | — |
| 6 | 21.4 | 8 | 9.8 | 70 |
| 7 | 10.0 | 10 | 9.4 | 80 |
| Mean ± SE | 13.0 ± 1.7 | 11.7 ± 1.0 | 10.8 ± 0.6 | 74.4 ± 2.6 (5) |

### EXAMPLE 6

The effects of compound 1 on kainate-induced IL-1β production was also studied as described in Example 1. IL-1β production was assessed by Western blot analysis of hippocampal homogenates obtained from rats 90 minutes after intrahippocampal kainate (40 ng) microinjection, as was ICE/caspase-1 activation. Total proteins (170 μg) from hippocampal homogenates were separated using SDS PAGE, 10% acrylamide and transferred to Hybond nitrocellulose membrane by electroblotting. ICE/Caspase-1 and IL-1β immunoreactivity was evaluated using selective antibodies and detected with enhanced chemiluminescence. Intrahippocampal kainate injection induced the formation of the active 20 kD subunit of ICE/caspase-1 and the formation of active 17 kD IL-1β. Compound 1, injected intracerebroventricularly (25 μg/4 μL), inhibited the activation of ICE/caspase-1, as evidenced by abolition of the formation of the active 20 kD subunit of ICE/caspase-1, and also reduced the formation mature active 17 kD IL-1β (see FIG. 1 and FIG. 2A for caspase-1 data and FIG. 2B for IL-1β data).

### EXAMPLE 7

Tablet Formation

Compound 2 may be formulated for oral administration as described below and in Table 6. The drug product was formulated to provide 300 mg of compound 2 per tablet.

TABLE 6
Composition of compound 2, 300 mg tablets

| | Quantity | |
|---|---|---|
| Component | (mg/tablet) | Function |
| Compound A | 300 | Active Ingredient |
| Microcrystalline Cellulose (NF) | 277.50 | Filler |
| Pregelatinized Starch (NF) | 131.25 | Disintegrant |
| Sodium Starch Glycolate (NF) | 15.00 | Disintegrant |
| Colloidal Silicon Dioxide (NF) | 11.25 | Glidant |
| Talc (USP) | 7.50 | Glidant |
| Magnesium Stearate (NF) | 7.50 | Lubricant |
| Total | 750 | |

### REFERENCES

A. Vezzani et al., “Powerful Anticonvulsant Action of IL-Receptor Antagonist on Intracerebral Injection and Astrocytic Overexpression in Mice” PNAS, 97, pp. 11534-11539 (2000).
B. Viviani et al. “Interleukin-1β Enhances NMDA Receptor-Mediated Intracellular Calcium Increase through Activation of the Src Family of Kinases” J. Neurosci., 23, pp. 8692-8700 (2003).
M. Rizzi et al., “Glia Activation and Cytokine Increase in Rat Hippocampus by Kainic Acid-induced Status Epilepticus During Postnatal Development” 14, pp. 494-503 (2003).
De Simoni et al., “Inflammatory Cytokines and Related Genes and Induced in the Rat Hippocampus by Limbic Status Epilepticus” 12, pp. 2623-2633 (2000).
A. Vezzani et al., “Interleukin-1β Immunoreactivity and Microglia are Enhanced in the Rat Hippocampus by Focal Kainate Application: Functional Evidence for Enhancement of Electrographic Seizures” J. Neurosci. 19, pp. 5054-5065 (1999).

All documents cited herein are hereby incorporated by reference.

While a number of embodiments of this invention have been described, it is apparent that the basic examples may be altered to provide other embodiments, which utilize the compounds and methods of this invention. Therefore, it will be appreciated that the scope of this invention is to be defined by the appended claims rather than by the specific embodiments, which have been represented by way of example.

We claim:

1. A method for treating seizures in a patient, comprising administering to the patient a compound that inhibits ICE/caspase-1.

2. A method for treating convulsions in a patient, comprising administering to the patient a compound that inhibits ICE/caspase-1.

3. A method for treating epilepsy in a patient, comprising administering to the patient a compound that inhibits ICE/caspase-1.

4. A method for preventing a seizure disorder in a patient, comprising administering to the patient a compound that inhibits ICE/caspase-1.

5. The method according to any one of claims 1-4, wherein the compound inhibits ICE/caspase-1 and one or more other caspase enzymes.

6. The method according to any one of claims 1-5, wherein the compound is a selective ICE/caspase-1 inhibitor.

7. The method according to any one of claims 1-6, wherein the compound is according to any of WO 95/35308, WO 97/22619, WO 99/47545, and WO 01/90063.

8. The method according to any one of claims 1-6, wherein the compound is: 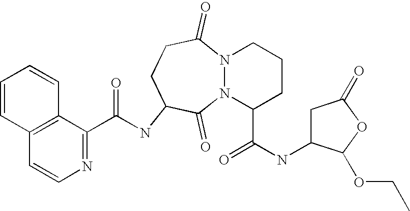
or any stereoisomer thereof, including: 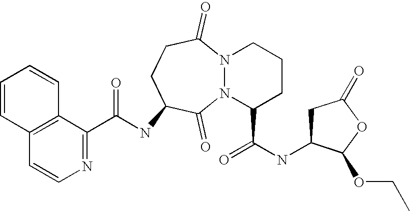
(compound 1).

9. The method according to any one of claims 1-6, wherein the compound is: 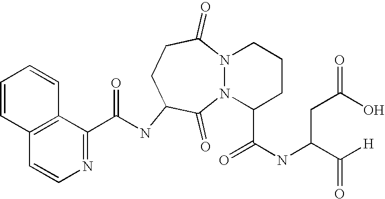
or any stereoisomer thereof, including:

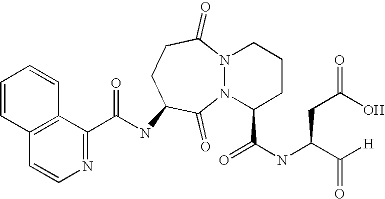

10. The method according to any one of claims 1-6, wherein the compound is: 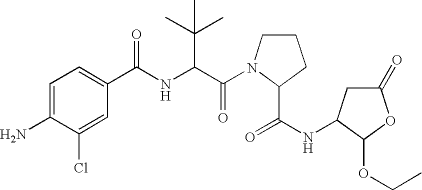
or any stereoisomer thereof, including: 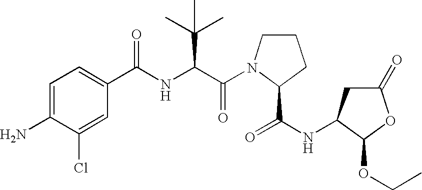
compound 2

11. The method according to any one of claims 1-6, wherein the compound is: 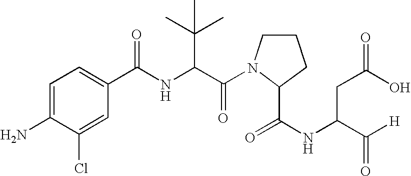
or any stereoisomer thereof, including:

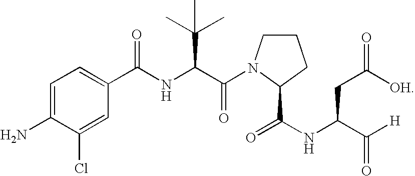

12. The method according to any one of claims 1-11, wherein the compound is administered peripherally (i.e., orally or parenteraly, not intracranially).

13. The method according to any one of claims 1-12 further comprising administering an additional compound, wherein the additional compound is an anticonvulsant compound.

14. The method according to claim 13, wherein the additional compound is mephobarbital, pentobarbital, ldrazepam, clonazepam, clorazepate, diazepam, tiagabin, gabapentin, pregabalin, vigabatrin, hydantoins, phenyloin, fosphenyloin, lamotrigine, methsuximide, ethosuximide, carbamazepine, riluzole, valproate, divalproex, felbamate, primidone, or topiramate.

15. A pharmaceutical composition for ameliorating, treating, or preventing seizures, convulsions, or epilepsy in a patient, comprising a compound that inhibits ICE/caspase-1 and a pharmaceutically acceptable carrier.

16. The pharmaceutical composition according to claim 15, wherein the composition further comprises another anticonvulsant compound.

17. The pharmaceutical composition according to claim 16, wherein the additional compound is mephobarbital, pentobarbital, lorazepam, clonazepam, clorazepate, diazepam, tiagabin, gabapentin, pregabalin, vigabatrin, hydantoins, phenyloin, fosphenyloin, lamotrigine, methsuximide, ethosuximide, carbamazepine, riluzole, valproate, divalproex, felbamate, primidone, or topiramate.

18. A kit comprising a compound that inhibits ICE and instructions for treating seizures, convulsions, or epilepsy using the compound.

19. The pharmaceutical composition according to any one of claims 15-17 or the kit according to claim 18, wherein the compound is as disclosed in any one WO 95/35308, WO 97/22619, WO 99/47545, or WO 01/90063 or as recited in claims 8-11.

20. An assay for identifying a compound for use in the treatment of seizures, convulsions, or epilepsy, comprising determining the ability of the compound to inhibit ICE/caspase-1.

21. An assay for identifying an ICE/caspase 1 inhibitor having anti-seizure, anti-convulsant, or anti-epileptic activity, comprising determining the ability of the ICE/caspase-1 inhibitor to inhibit seizures, convulsions, or epilepsy.

22. The assay according to claim 20 or claim 21, wherein the assay is done by methods substantially as described herein.
